(19) United States
(12) Patent Application Publication
(10) Pub. No.: US 2024/0050267 A1
(43) Pub. Date: Feb. 15, 2024

(54) POWERED INTRAORAL APPARATUS

(71) Applicant: Gwo-Tzong Hwang, Bethesda, MD (US)

(72) Inventor: Gwo-Tzong Hwang, Bethesda, MD (US)

(21) Appl. No.: 17/990,850

(22) Filed: Nov. 21, 2022

Related U.S. Application Data
(63) Continuation-in-part of application No. 17/885,529, filed on Aug. 10, 2022.

(51) Int. Cl. A61F 5/56; A61M 21/02

(52) U.S. Cl. CPC A61F 5/566; A61M 21/02; A61F 2005/563; A61M 2021/0027

(57) ABSTRACT

An approach is disclosed for transmitting customizable audio signals through teeth. An intraoral apparatus is provided with a teeth movement detection element to detect teeth movement. The apparatus holds a state where the state is one of on and off. The apparatus includes a power source, an audio element, a control element, and a bone conduction element. A customizable audio signals is received by the audio element. When teeth movement is detected when the state is off, the customizable audio signals are transmitted through the bone conduction element for a period of time.

If an Application Data Sheet (ADS) has been filed for this application, it is incorporated by reference herein. Any applications claimed on the ADS for priority under 35 U.S.C.§§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

For purposes of the USPTO extra-statutory requirements, the present application constitutes a utility application related to and claims the benefit of priority from U.S. patent application Ser. No. 17/885,529 filed on Aug. 10, 2022.

### BACKGROUND

The present invention relates to the field of intraoral apparatus, more specifically to deliver customizable audio signals for a limited time to a user through teeth.

### SUMMARY

According to one embodiment of the invention, there is provided a method for sending customizable audio signals through teeth activated by teeth movement detection element. An intraoral apparatus is provided with a teeth movement detection element to detect teeth movement. The teeth movement detection element holds a state where the state is one of on and off. The intraoral apparatus includes a power source, an audio element, a control element, and a bone conduction element. Customizable audio signals are received by the audio element. When teeth movement is detected by the teeth movement detection element when the state is off, the customizable audio signal is transmitted through the bone conduction element for a period of time.

According to one embodiment of the invention, there is an intraoral apparatus configured to send customizable audio signals through teeth activated by a teeth movement detection element. The intraoral apparatus includes the teeth movement detection element to detect teeth movement. The teeth movement detection element holds a state where the state is one of on and off. The intraoral apparatus includes a power source, an audio element, a control element, and a bone conduction element. Customizable audio signals are received by the audio element. When teeth movement is detected by the teeth movement detection element when the state is off, the customizable audio signal is transmitted through the bone conduction element for a period of time.

The foregoing is a summary and thus contains, by necessity, simplifications, generalizations, and omissions of detail; consequently, those skilled in the art will appreciate that the summary is illustrative only and is not intended to be in any way limiting. Other aspects, inventive features, and advantages of the present invention will be apparent in the non-limiting detailed description set forth below.

### BRIEF DESCRIPTION OF THE DRAWINGS

The present invention may be better understood, and its numerous objects, features, and advantages made apparent to those skilled in the art by referencing the accompanying drawings, wherein:

FIG. 1 is a schematic operational flow of an embodiment of the invention;

FIG. 2 is a schematic depiction including various components of an embodiment of an apparatus supporting the operational flow in FIG. 1;

FIG. 3 is a rear-view sketch of the various components of an embodiment of the apparatus shown in FIG. 2;

FIG. 4 is a right-side view sketch of the various components of an embodiment of the apparatus shown in FIG. 2;

FIG. 5 is a schematic depiction including various components of an embodiment of an apparatus supporting the operational flow in FIG. 1;

FIG. 6 is a schematic depiction including various components of an embodiment of an apparatus supporting the operational flow in FIG. 1;

FIG. 7 is a schematic operational flow of an embodiment of the invention; and

FIG. 8 is a schematic depiction including various components of an embodiment of an apparatus supporting the operational flow in FIG. 7.

### DETAILED DESCRIPTION

The present invention relates to the field of intraoral apparatus configured to treat bruxism, that is, excessive grinding of the teeth or clenching of the jaw. According to National Institutes of Health studies, about eight percent of the general population suffer from nighttime teeth grinding, or sleep bruxism. Statistics from a study of bite force and state of dentition shows the strength of an adult human bite can range around 11 kilogram-force (KGF) to about 39 KGF. Data from a quantitative study of bite force during sleep associated bruxism has shown that the mean amplitude of detected bruxism events was 22.5 KGF and the highest amplitude of nocturnal bite force in individual subjects was 42.3 KGF. The major consequences of sleep bruxism are headaches, tooth wear, and complaints from bed partner due to grinding noises.

Various prior art approaches have been developed to treat bruxism. One type of the prior art includes different shapes of occlusal guards. Different shapes of occlusal guards provide some protection to the teeth from grinding, but they do not stop bruxism. Another type of prior art embodiments uses sensors to monitor bruxism. When bruxism is detected, an audio alarm is triggered to alert/wake up the user to stop the bruxism. However, audio alarms severely disrupt the user and his/her bed partner's sleep and negatively impact their health from lack of sleep. Yet another type of the prior art embodiments also uses sensors to monitor the patient for bruxism, and when the bruxism occurs, electric current, laughing gas, or some medicines are applied to stimulate/relax the oral muscles. These devices might be able to stop the bruxism but are cumbersome and cannot be easily self-administered at home.

In view of the shortcomings in the prior arts, there is an advantage of embodiments of the apparatus to treat bruxism facilitating self-administered treatment at home. In some embodiment, the apparatus monitors the user for bruxism. When the bruxism is detected, a bone conduction element, for example, but not limited to, a bone conductor transducer, is used to send some calming music or customizable audio signals preselected by the user. Support may be provided to allow for a comfortable amplification/vibration strength level pre-set by the user, to the user's teeth via occlusal guards. This calming music or audio may be of the user's choice. The audio input may be, for example, but not limited to, a radio station, a streaming music service, a purchased soundtrack, and the like. The music or audio input is received from the bone conduction transducer, which is more likely to interrupt and stop user's bruxism, without waking up the user or his/her bed partner.

Embodiments of the apparatus facilitate an improved treatment of bruxism. An embodiment may have a flexible fastener connecting two housings for the left side and right side of the teeth, each of the two housings may be integrated with an occlusal guard. Each occlusal guard may be integrated with a pressure switch or other sensor or sensors that enable detection of bruxism. The housing may be perpendicularly connected to the occlusal guard and sit in an oral vestibule, the area between the teeth and the cheeks. The housing may include a control element, for example, but not limited to, a circuit control element with logic control codes, a long bite circuit, a delay off circuit, an auto off circuit, an audio element, at least one bone conductor transducer, and a power supply (power source.) The apparatus may further include an audio memory element. The power supply may be, for example, but not limited to, a disposable battery, a rechargeable battery that can be recharged from external power sources, etc. In some embodiments, the power supply may use energy derived from the user. In an embodiment, the grinding of the teeth or jaw may be used to generate a current which could be used to charge a battery.

The occlusal guard provides protection to the teeth when bruxism occurs. In an embodiment, a pressure switch is connected to the circuit control element. The pressure switch can be pressed on or pressed off by the user consciously biting and holding down the occlusal guard for longer than some predetermined duration of time (long bite), or by the user's randomly biting down by the teeth grinding from a sleep bruxism (short bite). When the pressure switch is pressed on from an off state, all electronic components, including the circuit control element and the power supply, may be activated. When activated by a short bite, the power supply element fast charges a capacitor in the delay off circuit. When the capacitor is fully charged (either a specific charge up voltage is reached, or when the voltage reaches the thermal noise value of the capacitor, or some other measurement methods), the auto off circuit sets the pressure switch to off state. When the pressure switch is off, the capacitor is not charged. The delay off circuit slowly uses the energy stored in the capacitor to regulate the power supply to continue to provide needed power to all electronic components in the housing until the capacitor is fully drained, or the power supply is fully drained. The pressure switch is likely to be pressed multiple times during a bruxism event, the delay off and auto off circuits ensure that the customizable audio signals are delivered continuously during the bruxism event and will be stopped shortly after the bruxism event stops. When the pressure switch, is pressed and held for a predetermined duration of time (long bite), from an on state, the following occurs: 1) The pressure switch state is set to off. 2) The energy stored in the capacitor of the delay off circuit is fast drained completely. 3) All electronic components in the apparatus are deactivated to prevent the sending of the customizable audio through the teeth. When the pressure switch, is pressed and held for a predetermined duration of time (long bite), from an off state, the following occurs: 1) The pressure switch state is set to on. 2) The power supply is activated to enable the sending of the customizable audio through the teeth. 3) The capacitor of the delay off circuit is not charged.

The audio element, when activated, can receive audio signals in a variety of transmission means. Examples of customizable audio signals, may for example, include, but are not limited to, a mobile phone playing user selected calming music at a preferred volume level, for example, via Bluetooth®¹ wireless transmission, and sends the received customizable audio signals to the bone conductor transducer. Streaming audio support may be supplied by other devices and transmission via subscription or a radio frequency. ¹Bluetooth is a trademark of BLUETOOTH SIG, INC.

When activated, the bone conductor transducer receives the customizable audio signal from the audio element and sends it via vibration to the user's teeth. To increase the sound quality, the bone conductor transducer can be placed in a sealed chamber immediately adjacent to the occlusal guards and the sealed chamber may be filled with non-electricity-conductive material, for example, but not limited to, non-electricity-conductive silicone gel.

In an embodiment, the apparatus can further comprise an audio memory element. The audio memory element comprises a memory area, an audio player, and functions to upload and store the preferred customizable audio signals onto the memory area and sends the stored customizable audio signals to the bone conductor transducer. Instead of playing customizable audio signals from the audio element, a user can choose to play the previously uploaded customizable audio signals from the audio memory element using the apparatus by uploading and storing customizable audio signals in the audio memory element. If there are no customizable audio signals stored in the audio memory element, the apparatus will play the customizable audio signals from the audio element.

When sleep bruxism occurs, the introduction of the calming customizable audio signals at a comfortable volume is more likely to stop the bruxism without waking up the user or disturbing other persons in the surrounding area, such as a bed partner.

When a user is jogging or swimming, headphones, air pods, and earbuds may fall off from the user's ears or the user's head. Embodiments of the intraoral apparatus may be securely held inside the user's mouth minimizing the chance of falling off. In addition, by having the intraoral apparatus in the user's mouth, interference with the surrounding environment is minimized.

The occlusal guard provides protection to the teeth when bruxism occurs. The pressure switch is connected to the circuit control element. The pressure switch may be pressed on or pressed off responsive to the user's actions, such as, by consciously biting down the occlusal guard (long bite) or by the teeth grinding from a bruxism event (short bite).

In an embodiment, when activated by a short bite, the power supply element fast charges a capacitor in the delay off circuit. When the capacitor is fully charged, the auto off circuit sets the pressure switch to off state. There are multiple ways to determine when a capacitor is fully charged, for example, but not limited to, determining when a specific charge-up voltage is reached, determining when the voltage reaches the thermal noise value of the capacitor, etc. When the pressure switch state is off, the capacitor is not charged. The delay off circuit slowly uses the energy stored in the capacitor to regulate the power supply element to continue to provide needed power to all electronic components in the housing until the capacitor is fully drained. The pressure switch is likely to be pressed multiple times during a bruxism event, the delay off circuit and the auto off circuit ensure that the customizable audio signals are delivered continuously during the bruxism event and will be stopped after the bruxism event stops. In some embodiments, the customizable audio signals delivery is stopped in a limited time, for example, 5 minutes, after the bruxism event stops.

In an embodiment, no energy level buildup is needed to send the customizable audio signals. When teeth movement is detected and the state is off, the state is set to on, and the customizable audio signals are sent through the bone conduction element, for a period of time, for example, N minutes. In that scenario, the teeth movement detection element is suspended for N minutes and the customizable audio signals continue to be sent through the bone conduction element. After the N minutes have elapsed, the sending of the customizable audio signals is discontinued, and the teeth movement detection element capability is enabled in an off state. In an embodiment, when a long bite is detected by the teeth movement detection element and the state is off, the state is turned on and the power source is activated to enable sending of the customizable audio signals. In an embodiment, when a long bite is detected by the teeth movement detection element and the state is on, the state is turned off, and the energy level in the control unit is fast dissipated fully, and the sending of the customizable audio signals is discontinued.

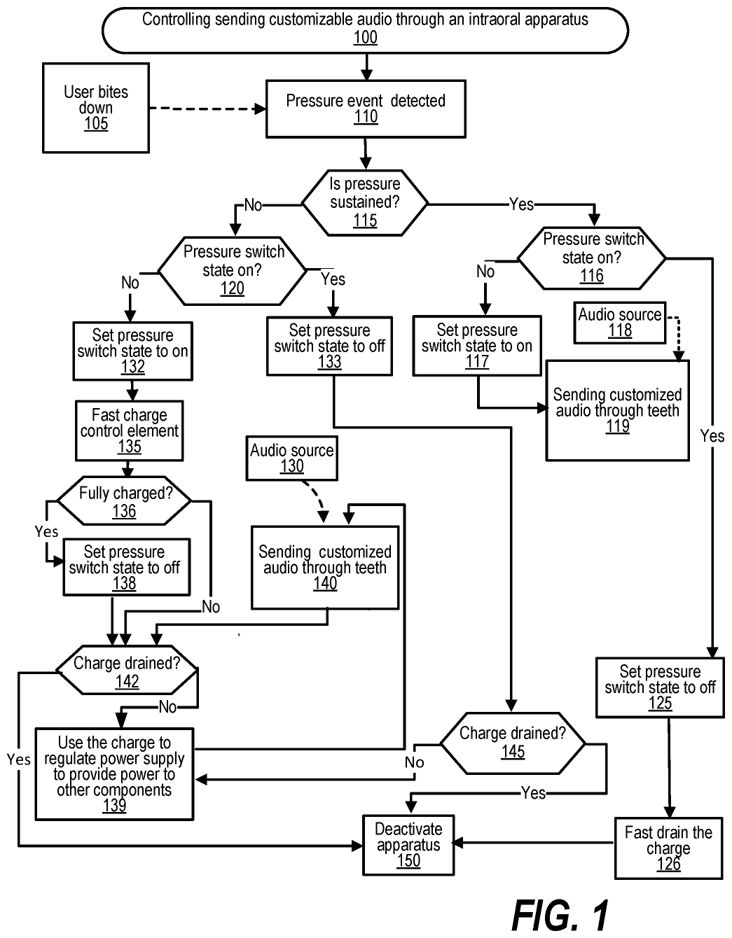

FIG. 1 processing commences at 100 and shows the steps taken by a process that controls sending customizable audio through an intraoral apparatus. At step 105, the user bites down on the pressure switch. The biting may be short bites due to grinding teeth, such as, occurs during bruxism. Alternatively, the user may be using long bites to intentionally issue commands that the device will execute based on how the device is configured. In some embodiments, a detection of a pressure exceeding a specific force may cause an action, such as, activating the device or deactivating the device. The amount of time the bite is held may be used to control details of the action. For example, a long bite, say exceeding 10 seconds, may mean immediate deactivation. A short bite, say under 3 seconds, may mean activate the device for a duration of time. At step 110, a pressure event is detected by the pressure switch or other sensor or sensors that may be used to detect teeth grinding or bruxism. A pressure event is detected responsive to a user biting down on the pressure switch or grinding teeth. Any bite on the pressure switch may trigger the pressure event detected by the pressure switch 110. If the grip of the teeth on the apparatus is released and the grinding restarts, a new pressure event may be detected. The process determines as to whether is pressure sustained (decision 115). If pressure is sustained (long bite), then decision 115 branches to the ‘Yes’ branch. If pressure is not sustained (short bite), then decision 115 branches to the ‘No’ branch. When the pressure is first detected, the process may, for example, set a stopwatch used to monitor how long the pressure is sustained. When the pressure switch detects an absence of pressure, the stopwatch is checked to see how long the pressure was maintained. If the stopwatch indicates a predetermined duration of time has elapsed, then decision 115 branches to ‘Yes’ branch (long bite). Alternatively, or additionally, when the pressure is first detected, the process may, for example, set a timer. If the timer triggers an interrupt indicating a set predetermined duration of time has elapsed before the absence of pressure is detected, the process notes that the pressure is sustained and follows the ‘Yes’ branch for decision 115. The process determines if pressure switch state is on (decision 116). If pressure switch state is on, then decision 116 branches to the ‘Yes’ branch. If pressure switch state is not on, then decision 116 branches to the ‘No’ branch. At step 117, the pressure switch state is set to on. At step 119, sending customizable audio signals through teeth utilizing audio source 118. The customizable audio signals may have been previously downloaded to the audio source 118 or be transferred real-time. At step 125, the pressure switch state is set to off. At step 126, the process fast drains the charge. At step 150, the intraoral apparatus is deactivated. When decision 115 branches to ‘No’ branch (short bite), the process determines if pressure switch state is on (decision 120). If pressure switch state is on, then decision 120 branches to the ‘Yes’ branch. If pressure switch state is not on, then decision 120 branches to the ‘No’ branch. At step 132, the process sets the switch state to on. At step 133, the process sets the pressure switch state to off. At step 135, the process fast charges control element (fast charging continues when the switch state remains on). The audio source 130 may have been supplied, for example, by downloading an audio file via a wired connection. Alternatively, the audio source 130 may be delivered real-time from a smart phone, via a short-range wireless technology, such as, Bluetooth. The process determines as to whether the control element is fully charged (decision 136). If the control element is fully charged, then decision 136 branches to the ‘Yes’ branch. On the other hand, if control element is not fully charged, then decision 136 branches to the ‘No’ branch. At step 138, the pressure switch state is set to off. The process determines as to whether charge drained (decision 142). If charge drained, then decision 142 branches to the ‘Yes’ branch that goes to step 150, the intraoral apparatus is deactivated. On the other hand, if not charge drained, then decision 142 branches to the ‘No’ branch. The process determines as to whether charge drained (decision 145). If charge is not drained, then decision 145 branches to the ‘No’ branch which goes to 139. At step 139, the process uses the charge from the control element to regulate power supply to provide power to other components. From 139, the process proceeds to step 140, continuing to send customizable audio through teeth which loops back to decision 142. This looping continues until the charge is drained, at which point decision 142 branches to the ‘Yes’ branch exiting the loop.

In addition, in an embodiment, the apparatus can also be used, more generally, to receive audio signals in a more discreet way from the environment, including, but not limited to, listening to favorite music when jogging, swimming, or studying in library.

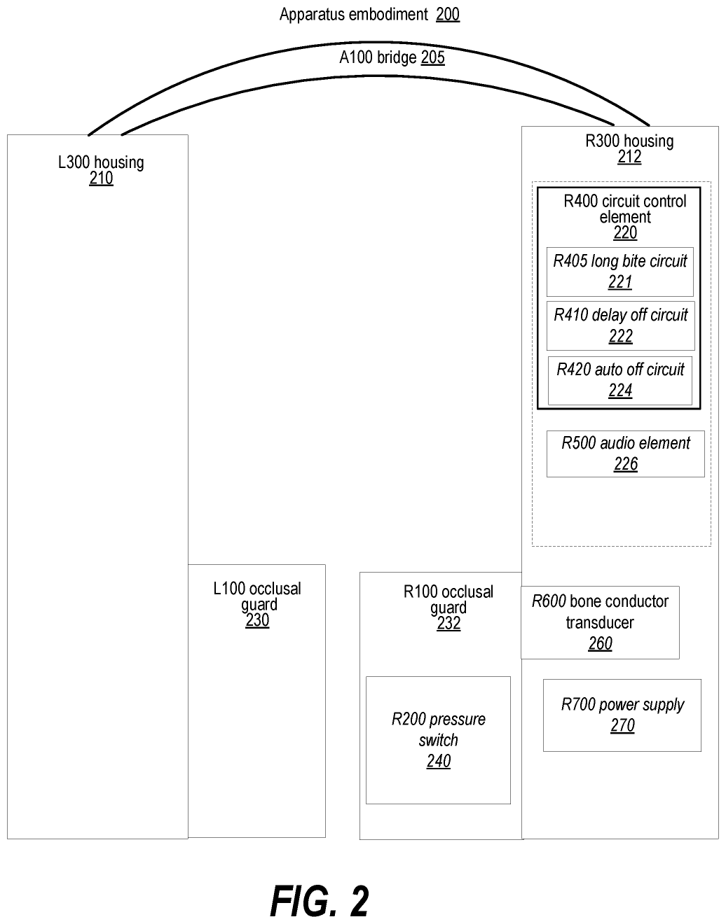

FIG. 2 is a schematic depiction including various components of an apparatus embodiment 200 supporting the operational flow in FIG. 1. The apparatus embodiment 200 may be shaped like some commonly available basic night guards. A A100 bridge 205 may be a flexible fastener that connects a L300 housing 210 for the left side of a mouth to a R300 housing 212 for the right side of the mouth. These housings may be fitted to be placed in the oral vestibule of the user. Each of the two housings may be integrated with corresponding occlusal guards. The L300 housing 210 is integrated with a L100 occlusal guard 230. The L300 housing 210 sits in the left side of the oral vestibule, that is, the area between the left teeth and the left cheek. The R300 housing 212 is integrated with a R100 occlusal guard 232. The R300 housing 212 sits in the right side of the oral vestibule, that is, the area between the right teeth and the right cheek. A R200 pressure switch 240 is integrated within the R100 occlusal guard 232 and connected to a R400 circuit control element 220 in the R300 housing 212. The R300 housing 212 houses the R400 circuit control element 220, a R500 audio element 226, a R600 bone conductor transducer 260, and a R700 power supply 270. The R400 circuit control element 220 may include a R405 long bite circuit 221, a R410 delay off circuit 222, and a R420 auto off circuit 224. The R600 bone conductor transducer 260 may be placed inside a sealed chamber immediately adjacent to the R100 occlusal guard 232 and filled with non-electricity-conductive silicone gel. The R400 circuit control element 220 has power circuits, data circuits, and process controlling logics that interconnects with and controls all other electronic components in the apparatus embodiment 200. The R410 delay off circuit 222 may comprise a capacitor. The R500 audio element 226 may comprise a Bluetooth module.

Before a regular use of the apparatus embodiment 200, the user may bite down the R200 pressure switch 240 to activate the apparatus embodiment 200, and pair the Bluetooth connection between the Bluetooth module in the audio element 226 of the apparatus embodiment 200 and a Bluetooth enabled audio playing device, for example, but not limited to, a mobile phone. The user may bite down the R200 pressure switch 240 again to turn off the apparatus embodiment 200 after the Bluetooth between the two devices are paired.

In a scenario for using the apparatus embodiment 200 to treat sleep bruxism. The user turns on the previously paired mobile phone with the Bluetooth function enabled, plays continuously his/her favorite calming audio with comfortable volume setting. The mobile phone can be placed in a place where the audio sound cannot be heard by the user, but its location is well within the Bluetooth connecting range.

The user then puts the apparatus embodiment 200 in his/her mouth, goes to bed, and falls asleep. If a sleep bruxism occurs and the user grinds his/her teeth, the teeth grinding force will turn the R200 pressure switch 240 state on. Once the switch state is on, the R405 long bite circuit 221 is activated to determine if the R200 pressure switch 240 is pressed and held for a predetermined duration of time or longer (long bite checking). The sleep bruxism normally would not bite down the teeth for a long duration of time, and in most cases, would fail the long bite checking and result as a short bite. When the switch is turned on by the short bite, a capacitor in the R410 delay off circuit 222 is fast charged by the R700 power supply 270.

When the R200 pressure switch 240 is pressed down while its state is on, the R405 long bite circuit 221 is activated to check if the pressed down is a long bite or short bite. A short bite triggers the R400 circuit control element 220 to set the R200 pressure switch 240 state to off and checks if the capacitor in the R410 delay off circuit 222 is fully drained. If the capacitor is fully drained, it deactivates the apparatus embodiment 200. If the capacitor is not fully drained, it continues to deliver the customizable audio signals to the user's teeth until the capacitor is fully drained, then it deactivates the apparatus embodiment 200. On the other hand, a long bite while the R200 pressure switch 240 state is on triggers the R400 circuit control element 220 to set the R200 pressure switch 240 state to off, fast drain the capacitor in the R410 delay off circuit 222, and deactivates the apparatus embodiment 200.

When power is supplied, the Bluetooth module in the R500 audio element 226 will be turned on and automatically pairs with another Bluetooth device, for example, a mobile phone. The mobile phone will automatically reroute the playing audio output to Bluetooth output. The audio received by R500 audio element 226 will then be routed to the R600 bone conductor transducer 260. The R600 bone conductor transducer 260 then delivers the calming customizable audio by vibration means via the R100 occlusal guard 232 to the user.

When the capacitor is fully charged, the R420 auto off circuit 224 sets the R200 pressure switch 240 to the off state. When the R200 pressure switch 240 state is off, the capacitor is not charged. There are multiple ways to determine if the capacitor is fully charged, for example, but not limited to, a specific charge up voltage is reached, or, when the voltage reaches the thermal noise value of the capacitor, etc. The R410 delay off circuit 222 design supports usage of the energy stored in the capacitor to regulate the R700 power supply 270 to continue to provide needed power to all electronic components in the R300 housing until the capacitor is completely drained, or the R700 power supply 270 is fully drained. The R200 pressure switch 240 is likely to be pressed multiple times during a bruxism event and its state will be switched between on and off multiple times. The R410 delay off circuit 222 and the R420 auto off circuit 224 ensure that the customizable audio signals are delivered continuously during the bruxism event and is stopped some short duration of time after the bruxism event stops. The R500 audio element 226, when activated, can receive customizable audio signals via a variety of transmission means. The transmission means include, but not limited to, a mobile phone playing user selected calming music at a preferred volume level, via Bluetooth wireless transmissions, and sends the received the customizable audio signals to the R600 bone conductor transducer 260. When activated, the R600 bone conductor transducer 260 receives the customizable audio signals and sends it via vibration to the user's teeth. To increase the sound quality, the R600 bone conductor transducer 260 can be placed in a sealed chamber immediately adjacent to the R100 occlusal guard 232 and the sealed chamber can be filled with non-electricity-conductive silicone gel.

Users can also use the apparatus when jogging, swimming, or in a library to listen to favorite audio with minimal interference to and from the surrounding environment. In a scenario for using the apparatus embodiment 200 when jogging, the user bites and holds down the R200 pressure switch 240 for a longer duration of time, for example, but not limited to, 10 seconds, this activates the R405 long bite circuit 221 to check and confirm that it is a long bite. When the apparatus embodiment 200 is turned on by a long bite, the R700 power supply 270 directly provides the power to R400 circuit control element 220, R500 audio element 226, and R600 bone conductor transducer 260. When power is supplied, the Bluetooth module in the R400 circuit control element 220 is turned on and automatically pairs with the mobile phone. The mobile phone automatically reroutes the playing audio output to Bluetooth output. The audio received by R500 audio element 226 is then routed to the R600 bone conductor transducer 260. The R600 bone conductor transducer 260 then delivers the calming customizable audio signals by vibration means via the R100 occlusal guard 232 to the user. Because the capacitor in the R410 delay off circuit 222 is not charged under this scenario, the user can bite down the R200 pressure switch 240 again, either via a long bite or a short bite, to immediately deactivate the apparatus embodiment 200.

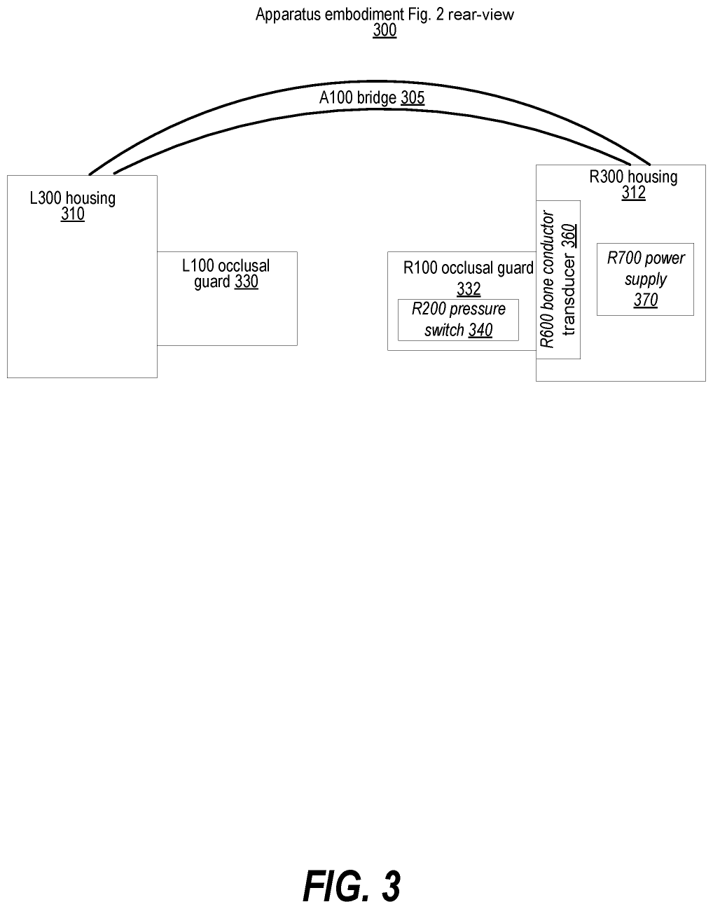

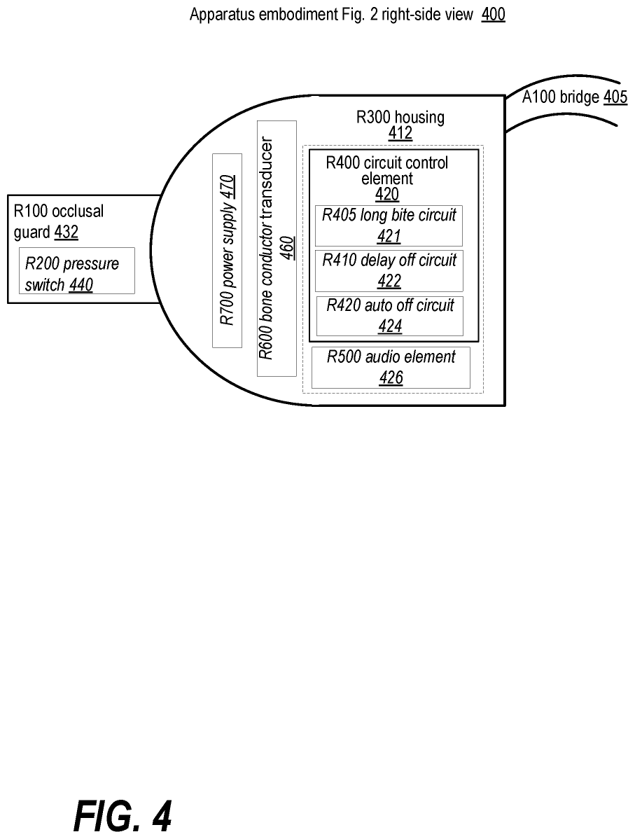

FIG. 3 and FIG. 4 display different view angles of the embodiment shown in FIG. 2 to provide better understanding of how the embodiment is used.

FIG. 3 is schematic depiction of the various components of the apparatus embodiment a rear-view 300 shown in FIG. 2. A A100 bridge 305 flexible fastener connects to a L300 housing 310 for the left side of a mouth and to a R300 housing 312 for the right side of the mouth. Each of the two housings may be integrated with corresponding occlusal guards. The L300 housing 310 is integrated with a L100 occlusal guard 330. The R300 housing 312 is integrated with a R100 occlusal guard 332. The housings may be placed in an oral vestibule, the area between the teeth and the cheeks. A R200 pressure switch 340 is integrated within the R100 occlusal guard 332. The R300 housing 312 houses a R600 bone conductor transducer 360 (can be placed inside a sealed chamber, immediately adjacent to the R100 occlusal guard 332, and filled with non-electricity-conductive silicone gel), and a R700 power supply 370.

FIG. 4 is schematic depiction of the various components of the apparatus embodiment right-side view 400 shown in FIG. 2. A A100 bridge 405 flexible fastener connects to a L300 housing for the left side of a mouth (not shown) and to a R300 housing 412 for the right side of the mouth. Each of the two housings may be integrated with corresponding occlusal guards. The R300 housing 412 houses a R400 circuit control element 420, a R500 audio element 426, a R600 bone conductor transducer 460, and a R700 power supply 470. The R400 circuit control element 420 may include a R405 long bite circuit 421, a R410 delay off circuit 422, and a R420 auto off circuit 424. A R200 pressure switch 440 is integrated within a R100 occlusal guard 432 and connected to the R400 circuit control element 420 in the R300 housing 412. The R600 bone conductor transducer 460 (may be placed inside a sealed chamber, immediately adjacent to the R100 occlusal guard 432, and filled with non-electricity-conductive silicone gel).

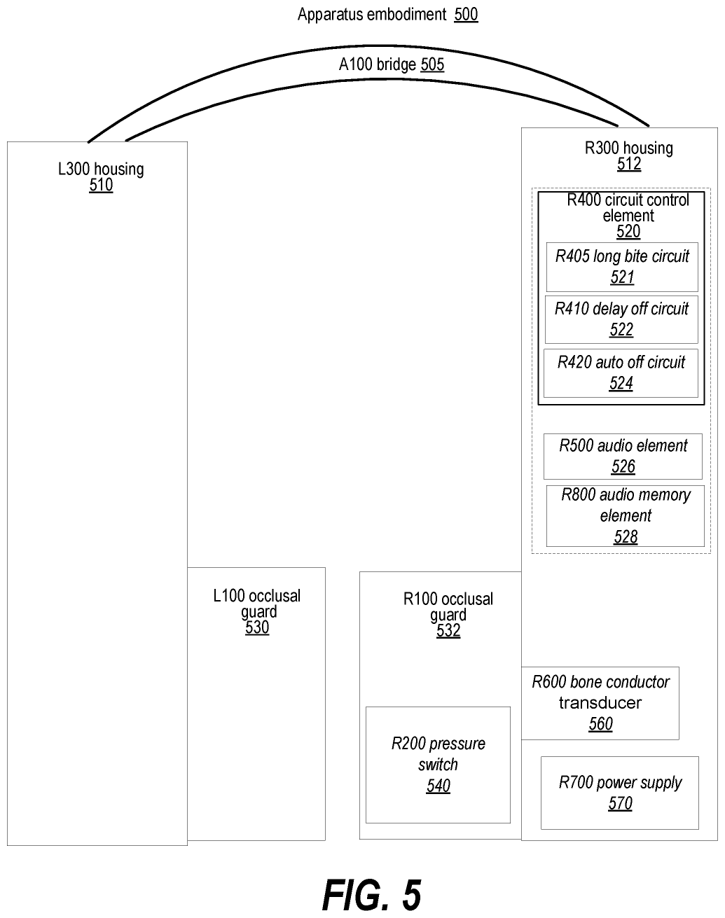

FIG. 5 is a schematic depiction including various components of another embodiment of an apparatus 500 supporting the operational flow in FIG. 1. The apparatus may be shaped like some commonly available basic night guards. This embodiment is like the embodiment in FIGS. 2-4, with an additional R800 audio memory element 528, with enhanced R500 circuit control element capacity, to enable the user to upload customizable audio signals to this embodiment to be played when this embodiment is turned on.

A A100 bridge 505 flexible fastener connects a L300 housing 510 for the left side of a mouth to a R300 housing 512 for the right side of the mouth. Each of the two housings may be integrated with corresponding occlusal guards. The L300 housing 510 is integrated with a L100 occlusal guard 530. The R300 housing 512 is integrated with a R100 occlusal guard 532. The housings 510 and 512 may be placed in an oral vestibule, the area between the teeth and the cheeks. A R200 pressure switch 540 is integrated within the R100 occlusal guard 532 and connected to a R400 circuit control element 520 in the R300 housing 512. The R300 housing 512 houses the R400 circuit control element 520, a R500 audio element 526, the R800 audio memory element 528, a R600 bone conductor transducer 560 (may be placed inside a sealed chamber, immediately adjacent to the R100 occlusal guard 532, and filled with non-electricity-conductive silicone gel), and a R700 power supply 570. The R400 circuit control element 520 may include a R405 long bite circuit 521, a R410 delay off circuit 522, and a R420 auto off circuit 524. In this embodiment, the R800 audio memory element 528 comprises a memory area, an audio codec, functions to upload and store some preferred customizable audio signals onto the memory area and sends the stored customizable audio signals to the R600 bone conductor transducer 560 to be delivered to the intraoral apparatus user. Instead of playing customizable audio signals from the R500 audio element 526, a user can choose to play the customizable audio signals stored in the R800 audio memory element 528. The R400 circuit control element 520 has power circuits, data circuits, and process controlling logics that interconnects with and controls all other electronic components in the apparatus embodiment 500. By uploading customizable audio signals onto the R800 audio memory element 528, the R400 circuit control element 520 will play and replay the stored customizable audio signals continuously to the user via the R600 bone conductor transducer 560. By erasing all stored customizable audio signals from the R800 audio memory element 528, the R400 circuit control element 520 will play the customizable audio signals from the R500 audio element 526 to the user via the R600 bone conductor transducer 560.

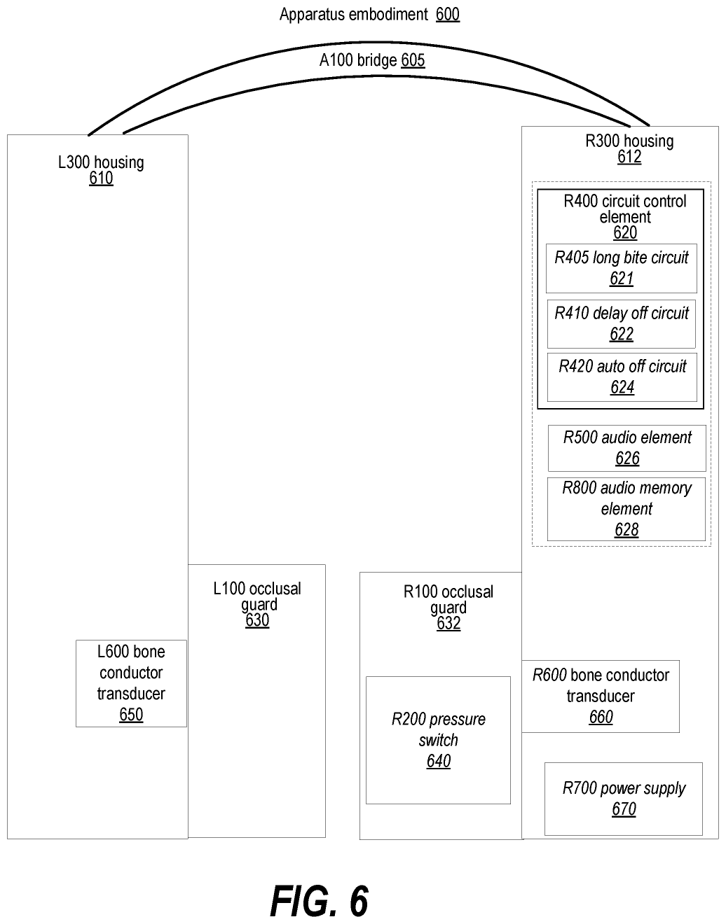

FIG. 6 is a schematic depiction including various components of an embodiment of an apparatus supporting the operational flow in FIG. 1, the apparatus embodiment 600. This embodiment is like the embodiment in FIG. 5, with an additional L600 bone conductor transducer 650 and an enhanced R400 circuit control element 620 with increased capacity to enable stereo effect audio to be played on both sides of the user's teeth.

The apparatus may be shaped like some of the commonly available basic night guards. A A100 bridge 605 flexible fastener connects a L300 housing 610 for the left side of a mouth to a R300 housing 612 for the right side of the mouth. Each of the two housings may be integrated with corresponding occlusal guards. The L300 housing 610 is integrated with a L100 occlusal guard 630. The R300 housing 612 is integrated with a R100 occlusal guard 632. The L300 housing 610 houses the L600 bone conductor transducer 650. The housings 610 and 612 may be placed in an oral vestibule, the area between the teeth and the cheeks. A R200 pressure switch 640 is integrated within the R100 occlusal guard 632 and connected to the R400 circuit control element 620 in the R300 housing 612. The R300 housing 612 houses the R400 circuit control element 620, a R500 audio element 626, a R600 bone conductor transducer 660 (may be placed inside a sealed chamber, immediately adjacent to the R100 occlusal guard 632, and filled with non-electricity-conductive silicone gel), a R700 power supply 670, and a R800 audio memory element 628. The R400 circuit control element 620 may include a R405 long bite circuit 621, a R410 delay off circuit 622, and a R420 auto off circuit 624. The R400 circuit control element 620 has power circuits, data circuits, and process controlling logics that interconnects with and controls all other electronic components in the apparatus embodiment 600. The L600 bone conductor transducer 650 can work with the R600 bone conductor transducer 660 to deliver the customizable audio signals in stereo to both sides of the user's teeth, creating stereo effect for the user's listening experience. In an embodiment, both sides may be used to maintain a stereo effect and provide an enhanced audio experience. In another embodiment, only one side may be implemented or enabled to allow a longer listening time. Options such as preloading the customized audio may also be used to allow for a longer listening time as opposed to streaming content which would require additional power.

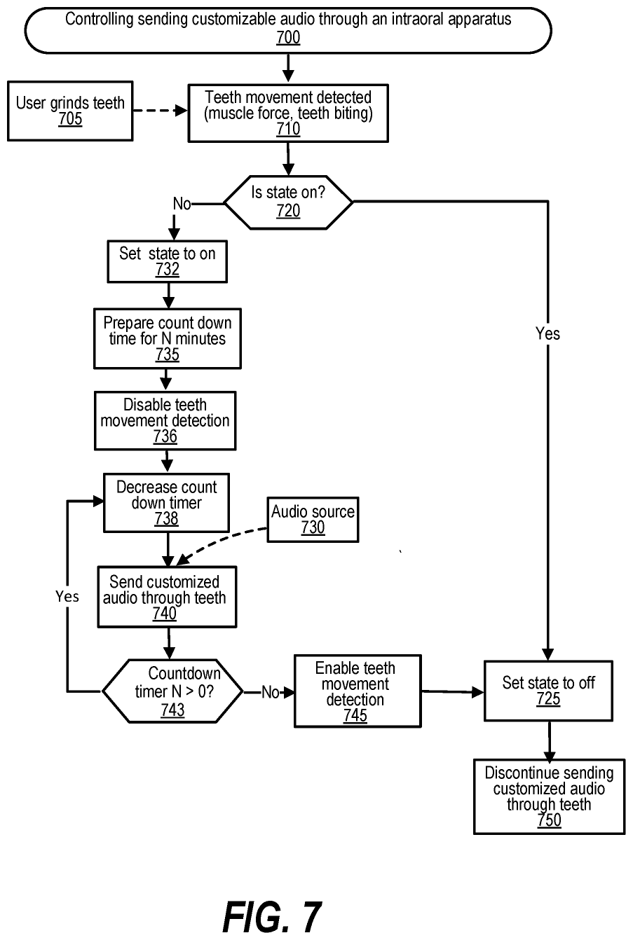

FIG. 7 processing commences at 700 and shows the steps taken by a process that controls sending customizable audio through an intraoral apparatus. At step 705, the user grinds teeth. The griding event may be short, such as, occurs during bruxism. Alternatively, the user may be using long bites to intentionally issue commands that the device will execute based on how the device is configured. In some embodiments, a detection of a pressure exceeding a specific force may cause an action, such as, activating the device or deactivating the device. The amount of time the bite is held may be used to control details of the action. For example, a long bite, say exceeding 10 seconds, may mean immediate change of state. If the device is deactivated, that is, not sending customized audio, the long bite may cause the device to send customized audio indefinitely. Alternatively, if the device is already sending customized audio through the teeth, the long bite may stop the sending of the customized audio through the teeth. The process may distinguish between a long bite and a short bite by various approaches. In an embodiment, when the pressure is first detected, the process may, for example, set a stopwatch used to monitor how long the pressure is sustained. When the pressure switch detects an absence of pressure, the stopwatch is checked to see how long the pressure was maintained. If the stopwatch indicates a predetermined duration of time has elapsed, then the process handles the event as a long bite. Alternatively, or additionally, when the pressure is first detected, the process may, for example, set a timer. If the timer triggers an interrupt indicating a set predetermined duration of time has elapsed before the absence of pressure is detected, the process notes that the pressure is sustained and handles the event as a long bite. In some embodiments, such as that shown in FIG. 7, there may be no distinction between a long bite and a short bite. A short bite, say less than 3 seconds, or any bite or teeth grinding may activate the device for a duration of time. At step 710, a teeth movement or pressure event is detected. The detection may be a specialized sensor or set of sensors to identify bruxism or a pressure switch. The teeth movement event may be detected responsive to a user biting down on the pressure switch or grinding teeth. Any bite on the pressure switch may trigger the teeth movement event. In an embodiment, the apparatus supports a state, where the state is one of on and off. If the grip of the teeth on the apparatus is released and subsequently the grip restarts again, a new pressure event may be detected. The process determines if state is on (decision 720). If pressure switch state is on, then decision 720 branches to the ‘Yes’ branch which goes to step 725 where the state is set to off, then the process continues to step 750 where the intraoral apparatus discontinues sending customized audio through the teeth, that is, deactivated. If the state is not on, then decision 720 branches to the ‘No’ branch which goes to step 732. At step 732, the process sets the switch state to on and goes to step 735. At step 735 a countdown timer is prepared to count down for N minutes. The process flow then continues to step 736 where the teeth movement detection is disabled. Then process flow then continues to step 738 where countdown timer is decreased. The process flow then continues to step 740. At step 740, the customizable audio signals are sent through the teeth utilizing an audio source 730. The audio source 730 may have been supplied, for example, by downloading an audio file via a wired connection. Alternatively, the audio source 730 may be delivered real-time from a smartphone, via a short-range wireless technology, such as Bluetooth. The process determines as to whether countdown timer N>0 (decision 743). If countdown timer N>0, then decision 743 branches to the ‘Yes’ branch which loops back to 738. This looping continues until countdown time N is no longer greater than 0 (N minutes have elapsed) at which point decision 743 branches to the ‘No’ branch exiting the loop to 745. At step 745, the teeth movement detection is enabled. The process flow continues to step 725 where the state is set to “Off”. The process flow will then continue to step 750 where the intraoral apparatus discontinues sending customize audio through teeth, that is, is deactivated.

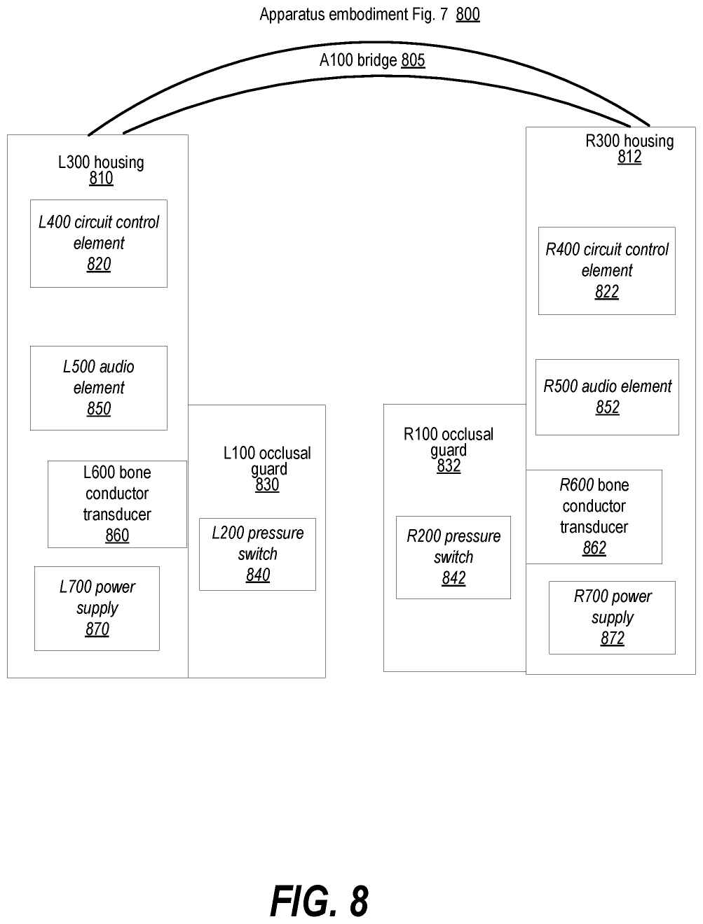

FIG. 8 is a schematic depiction including various components of an embodiment of an apparatus 800 supporting the operational flow in FIG. 7. The apparatus may be shaped like some commonly available basic night guards. A A100 bridge 805 flexible fastener connects a L300 housing 810 for the left side of a mouth to a R300 housing 812 for the right side of the mouth. Each of the two housings may be integrated with corresponding occlusal guards. The L300 housing 810 is integrated with a L100 occlusal guard 830. The L100 occlusal guard 830 is integrated with a L200 pressure switch 840. The R300 housing 812 is integrated with a R100 occlusal guard 832. The R100 occlusal guard 832 is integrated with a R200 pressure switch 842. The L300 housing 810 houses a L400 circuit control element 820, a L500 audio element 850, a L600 bone conductor transducer 860 (may be placed inside a sealed chamber, immediately adjacent to the L100 occlusal guard 830, and filled with non-electricity-conductive silicone gel), and a L700 power supply 870. The R300 housing 812 houses a R400 circuit control element 822, a R500 audio element 852, a R600 bone conductor transducer 862 (may be placed inside a sealed chamber, immediately adjacent to the R100 occlusal guard 832, and filled with non-electricity-conductive silicone gel), and a R700 power supply 872. The housings 810 and 812 may be placed in an oral vestibule, the area between the teeth and the cheeks. Both the L400 circuit control element 820 and the R400 circuit control element 822 have power circuits, data circuits, and process controlling logics that interconnects with and controls all other electronic components in the apparatus embodiment 800. The L600 bone conductor transducer 860 can work with the R600 bone conductor transducer 862 to deliver the customizable audio signals in stereo to both sides of the user's teeth, creating stereo effect for the user's listening experience.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While particular embodiments have been shown and described, it will be obvious to those skilled in the art that, based upon the teachings herein, that changes and modifications may be made without departing from this invention and its broader aspects. Therefore, the appended claims are to encompass within their scope all such changes and modifications as are within the true spirit and scope of this invention. Furthermore, it is to be understood that the invention is solely defined by the appended claims. It will be understood by those with skill in the art that if a specific number of an introduced claim element is intended, such intent will be explicitly recited in the claim, and in the absence of such recitation no such limitation is present. For non-limiting example, as an aid to understanding, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to inventions containing only one such element, even when the same claim includes the introductory phrases “one or more” or “at least one” and indefinite articles such as “a” or “an”; the same holds true for the use in the claims of definite articles

The possible usages of this apparatus are not limited to the embodiments described above, as additional embodiments of this apparatus are also possible.

What is claimed is:

1. A method for transmitting customizable audio signals through teeth activated by teeth movement comprising:
providing an intraoral apparatus with a teeth movement detection element to detect teeth movement that holds a state wherein the state is one of on and off, a power source, an audio element, a control element, and a bone conduction element;
receiving by the audio element the customizable audio signals; and
responsive to detecting teeth movement by the teeth movement detection element when the state is off, transmitting the customizable audio signals through the bone conduction element for a period of time.

2. The method of claim 1, wherein the period of time is adjustable.

3. The method of claim 1, further comprising:
building up an energy level in the control element when the state is on to regulate the power source used for transmitting of the customizable audio signals.

4. The method of claim 3, further comprising:
dissipating the energy level when regulating the power source; and
preventing, automatically, the transmitting of the customizable audio signals when the energy level is fully dissipated.

5. The method of claim 3, further comprising:
monitoring the building up of the energy level for a completion of build up; and
responsive to detecting the completion of the buildup of the energy level, setting the state to off.

6. The method of claim 3, further comprising:
responsive to detecting the received teeth movement extended over a predetermined duration of time while the state is on, setting the state to off, and fast dissipating the energy level completely.

7. The method of claim 3, further comprising:
responsive to detecting the received teeth movement extended over a predetermined duration of time while the state is off,
bypassing the building up of the energy level, setting the state to on, and enabling the power source to provide power to transmit the customizable audio signals.

8. The method of claim 1, wherein the method is used for treating bruxism and further comprises:
minimizing likelihood of waking up a user.

9. The method of claim 1, wherein the method allows a user to listen to the customizable audio signals with minimal interference from outside noise and with minimal interference to surrounding environment.

10. An intraoral apparatus configured to transmit customizable audio signals through teeth activated by teeth movement wherein the intraoral apparatus further comprises:
a teeth movement detection element to detect teeth movement that holds a state wherein the state is one of on and off, a power source, an audio element, a control element, and a bone conduction element;
receiving, by the audio element, the customizable audio signals; and
responsive to detecting teeth movement by the teeth movement detection element when the state is off, transmitting the customizable audio signals through the bone conduction element for a period of time.

11. The intraoral apparatus of claim 10, wherein the period of time is adjustable.

12. The intraoral apparatus of claim 10, further comprising:
building up an energy level in the control element when the state is on to regulate the power source used for transmitting of the customizable audio signals.

13. The intraoral apparatus of claim 12, further comprising:
dissipating the energy level when regulating the power source; and
preventing, automatically, the transmitting of the customizable audio signals when the energy level is fully dissipated.

14. The intraoral apparatus of claim 12, further comprising:
monitoring the building up of the energy level for a completion of build up; and
responsive to detecting the completion of the buildup of the energy level, setting the state to off.

15. The intraoral apparatus of claim 12, further comprising:
responsive to detecting the received teeth movement extended over a predetermined duration of time while the state is on, setting the state to off, and fast dissipating the energy level completely.

16. The intraoral apparatus of claim 12, further comprising:
responsive to detecting the received teeth movement extended over a predetermined duration of time while the state is off,
bypassing the building up of the energy level, setting the state to on, and enabling the power source to provide power to transmit the customizable audio signals.

17. The intraoral apparatus of claim 10, wherein the intraoral apparatus is used for treating bruxism and minimizing likelihood of waking up a user.

18. The intraoral apparatus of claim 10, wherein the intraoral apparatus allows a user to listen to the customizable audio signals with minimal interference from outside noise and with minimal interference to surrounding environment.

19. The intraoral apparatus of claim 10, wherein the bone conduction element further comprises: a left bone conduction element and a right bone conduction element.

20. The intraoral apparatus of claim 10, further comprising:
a left occlusal guard and a right occlusal guard and wherein at least one of the left occlusal guard and the right occlusal guard is integrated with the teeth movement detection element.
